(19) United States
(12) Patent Application Publication
(10) Pub. No.: US 2016/0038435 A1
(43) Pub. Date: Feb. 11, 2016

(54) TRANSDERMAL FORMULATIONS OF LAQUINIMOD

(71) Applicants: Ralph STEFAN, Ebenweiler (DE); Hans-Juergen MIKA, (US); Tanja PRIES, Munich (DE); Dirk SCHENK, Dietramszell (DE); Sabine PROHL, Munich (DE)

(72) Inventors: Ralph STEFAN, Ebenweiler (DE); Hans-Juergen MIKA, Bonn (DE); Tanja PRIES, Munich (DE); Dirk SCHENK, Dietramszell (DE); Sabine PROHL, Munich (DE)

(73) Assignee: Teva Pharmaceutical Industries Ltd., Petach Tikva (IR)

(21) Appl. No.: 14/773,658

(22) PCT Filed: Mar. 13, 2014
(86) PCT No.: PCT/US2014/026807

§ 371 Date: Sep. 8, 2015

Related U.S. Application Data
(60) Provisional application No. 61/781,585, filed on Mar. 14, 2013.

(51) Int. Cl. A61K 9/70; A61K 47/14; A61K 47/12; A61K 31/4704; A61K 47/10

(52) U.S. Cl. CPC A61K 9/7061; A61K 31/4704; A61K 47/10; A61K 47/12; A61K 47/14

(57) ABSTRACT

This invention provides a transdermal patch comprising a) a backing layer; b) a liner; c) optionally, a highly porous membrane; and d) a pharmaceutical composition comprising: (i) optionally, a pressure sensitive adhesive in an amount of up to about 95 wt % of the pharmaceutical composition, (ii) laquinimod in an amount of about 0.1-20 wt % of the pharmaceutical composition, and (iii) optionally, one or more permeation enhancers in a total amount of up to about 70 wt % of the pharmaceutical composition. This invention also provides a method for delivering laquinimod across the skin of the subject and for treating a human subject afflicted with a form of multiple sclerosis comprising administering to the skin of the subject a transdermal patch as described herein. This invention further provides a transdermal patch as described herein for use in treating a human subject afflicted with a form of multiple sclerosis.

This application claims priority of U.S. Provisional Application No. 61/781,585, filed Mar. 14, 2013, the entire content of which is hereby incorporated by reference herein.

Throughout this application, various publications are referred to by first author and year of publication. Full citations for these publications are presented in a References section immediately before the claims. Disclosures of the documents and publications cited are hereby incorporated by reference in their entireties into this application in order to more fully describe the state of the art as of the date of the invention described herein.

### BACKGROUND

### Multiple Sclerosis

Multiple Sclerosis (MS) is a chronic, debilitating disease of the central nervous system (CNS). MS has also been classified as an autoimmune disease. MS disease activity can be monitored by magnetic resonance imaging (MRI) of the brain, accumulation of disability, as well as rate and severity of relapses.

There are five main forms of multiple sclerosis:

### 1) Benign Multiple Sclerosis:

Benign multiple sclerosis is a retrospective diagnosis which is characterized by 1-2 exacerbations with complete recovery, no lasting disability and no disease progression for 10-15 years after the initial onset. Benign multiple sclerosis may, however, progress into other forms of multiple sclerosis.

### 2) Relapsing-Remitting Multiple Sclerosis (RRMS):

Patients suffering from RRMS experience sporadic exacerbations or relapses, as well as periods of remission. Lesions and evidence of axonal loss may or may not be visible on MRI for patients with RMS.

### 3) Secondary Progressive Multiple Sclerosis (SPMS):

SPMS may evolve from RRMS. Patients afflicted with SPMS have relapses, a diminishing degree of recovery during remissions, less frequent remissions and more pronounced neurological deficits than RRMS patients. Enlarged ventricles, which are markers for atrophy of the corpus callosum, midline center and spinal cord, are visible on MRI of patients with SPMS.

### 4) Primary Progressive Multiple Sclerosis (PPMS):

PPMS is characterized by a steady progression of increasing neurological deficits without distinct attacks or remissions. Cerebral lesions, diffuse spinal cord damage and evidence of axonal loss are evident on the MRI of patients with PPMS.

### 5) Progressive-Relapsing Multiple Sclerosis (PRMS):

PRMS has periods of acute exacerbations while proceeding along a course of increasing neurological deficits without remissions. Lesions are evident on MRI of patients suffering from PRMS (Multiple sclerosis: its diagnosis, symptoms, types and stages, 2003).

Chronic progressive multiple sclerosis is a term used to collectively refer to SPMS, PPMS, and PRMS (Types of Multiple Sclerosis (MS), 2005). The relapsing forms of multiple sclerosis are SPMS with superimposed relapses, RRMS and PRMS.

A clinically isolated syndrome (CIS) is a single monosymptomatic attack compatible with MS, such as optic neuritis, brain stem symptoms, and partial myelitis. Patients with CIS that experience a second clinical attack are generally considered to have clinically definite multiple sclerosis (CDMS). Over 80 percent of patients with a CIS and MRI lesions go on to develop MS, while approximately 20 percent have a self-limited process (Frohman et al., 2003).

Multiple sclerosis may present with optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning.

### Laquinimod

Laquinimod is a novel synthetic compound with high oral bioavailability which has been suggested as an oral formulation for the treatment of Multiple Sclerosis (MS) (Polman, 2005; Sandberg-Wollheim, 2005). Laquinimod and its sodium salt form are described in, for example, U.S. Pat. No. 6,077,851.

The mechanism of action of laquinimod is not fully understood. Animal studies show it causes a Th1 (T helper 1 cell, produces pro-inflammatory cytokines) to Th2 (T helper 2 cell, produces anti-inflammatory cytokines) shift with an anti-inflammatory profile (Yang, 2004; Brück, 2011). Another study demonstrated (mainly via the NFkB pathway) that laquinimod induced suppression of genes related to antigen presentation and corresponding inflammatory pathways (Gurevich, 2010).

Laquinimod showed a favorable safety and tolerability profile in two phase III trials (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

### SUMMARY OF THE INVENTION

This invention provides a transdermal patch comprising: a) a backing layer; b) a liner; c) optionally, a highly porous membrane; and d) a pharmaceutical composition comprising: (i) optionally, a pressure sensitive adhesive in an amount of up to about 95 wt % of the pharmaceutical composition, (ii) laquinimod in an amount of about 0.1-20 wt % of the pharmaceutical composition, and (iii) optionally, one or more permeation enhancers in a total amount of up to about 70 wt % of the pharmaceutical composition.

This invention provides a method for delivering laquinimod across the skin of a subject comprising administering to the skin of the subject a transdermal patch as described herein.

This invention provides a method for treating a human subject afflicted with a form of multiple sclerosis, comprising periodically administering to the human subject a transdermal patch as described herein.

This invention provides a transdermal patch as described herein for use in treating a human subject afflicted with a form of multiple sclerosis.

### BRIEF DESCRIPTION OF THE DRAWINGS

FIG. 1: Differential Scanning calorimetry (DSC) Results from Experiment 1.

FIG. 2: The Diffusion Cell Assembly (Franz-Cell) referenced in the Experiments section.

FIG. 3: Skin permeation of laquinimod sodium from saturated solutions (mouse skin).

FIG. 4: Skin permeation of laquinimod free acid from saturated solutions (mouse skin).

FIG. 5: Skin permeation of laquinimod free acid from TTS in hairless mouse skin.

FIG. 6: Skin permeation (human skin vs. mouse skin) for laquinimod free acid from TTS.

FIG. 7: Comparison of in vitro skin permeation test results through hairless mouse skin from two batches containing different drug concentrations and different enhancers.

FIG. 8: Skin permeation results of a reservoir patch comprising laquinimod sodium.

FIG. 9: Schematic design of a reservoir patch according to the present invention.

### DETAILED DESCRIPTION OF THE INVENTION

This invention provides a transdermal patch comprising: a) a backing layer; b) a liner; c) optionally, a highly porous membrane; and d) a pharmaceutical composition comprising: (i) optionally, a pressure sensitive adhesive in an amount of up to about 95 wt % of the pharmaceutical composition, (ii) laquinimod in an amount of about 0.1-20 wt % of the pharmaceutical composition, and (iii) optionally, one or more permeation enhancers in a total amount of up to about 70 wt % of the pharmaceutical composition.

In one embodiment, the pharmaceutical composition is in the form of a layer. In another embodiment, the pharmaceutical composition is in the form of a film. In another embodiment, the pharmaceutical composition is in the form of a liquid.

In one embodiment, the transdermal patch further comprises a highly porous membrane. In another embodiment, the pharmaceutical composition further comprises a pressure sensitive adhesive in an amount of up to about 95 wt % of the pharmaceutical composition. In another embodiment, the pharmaceutical composition comprises one or more permeation enhancers present in a total amount of up to about 70 wt % of the pharmaceutical composition.

In one embodiment, the transdermal patch is a matrix patch. In another embodiment, the matrix patch comprises a pressure sensitive adhesive in an amount of up to about 95 wt % of the pharmaceutical composition. In another embodiment, the matrix patch comprises one or more permeation enhancers in a total amount of up to about 20 wt % of the pharmaceutical composition.

In an embodiment, the transdermal patch is a reservoir patch. In another embodiment, the reservoir patch further comprises a highly porous membrane. In another embodiment, the reservoir patch comprises one or more permeation enhancers in a total amount of up to about 70 wt % of the pharmaceutical composition.

In one embodiment, laquinimod is laquinimod free acid. In another embodiment, laquinimod is laquinimod sodium.

In one embodiment, the amount of laquinimod present in the pharmaceutical composition is a least laquinimod's saturation amount. In another embodiment, the amount of laquinimod present in the pharmaceutical composition is higher than laquinimod's saturation amount.

In one embodiment, laquinimod is present in an amount of about 1-15 wt % of the pharmaceutical composition. In another embodiment, laquinimod is present in an amount of about 2-10 wt % of the pharmaceutical composition. In another embodiment, laquinimod is present in an amount of about 1 wt % of the pharmaceutical composition. In another embodiment, laquinimod is present in an amount of 1 wt % of the pharmaceutical composition. In another embodiment, laquinimod is present in an amount of about 3 wt % of the pharmaceutical composition. In another embodiment, laquinimod is present in an amount of about 3.3 wt % of the pharmaceutical composition. In another embodiment, laquinimod is present in an amount of about 6.0 wt % of the pharmaceutical composition. In yet another embodiment, the amount of laquinimod present in the pharmaceutical composition is at least about 6.0 wt %.

In one embodiment, the transdermal patch contains about 0.1-20 mg laquinimod. In another embodiment, the transdermal patch contains about 0.1-10 mg laquinimod. In another embodiment, the transdermal patch contains about 6-8 mg laquinimod. In another embodiment, the transdermal patch contains about 7 mg laquinimod.

In one embodiment, the pressure sensitive adhesive is present in an amount of about 80-95 wt % of the pharmaceutical composition. In another embodiment, the pressure sensitive adhesive comprises an acrylate copolymer.

In another embodiment, the one or more permeation enhancers is present in a total amount of up to about 20 wt % of the pharmaceutical composition. In another embodiment, the one or more permeation enhancers is present in a total amount of up to about 15 wt % of the pharmaceutical composition. In another embodiment, the one or more permeation enhancers is selected from the group consisting of a fatty acid, an alcohol, diethylene glycol monoethyl ether, alpha-tocopherol, a sulfoxyde, an azone, a pyrrolidone or a derivative thereof, a terpene, a terpenoide, methyl acetate, butyl acetate and a cyclodextrine. In another embodiment, at least one of the one or more permeation enhancers is oleic acid. In another embodiment, at least one of the one or more permeation enhancers is isopropyl myristate. In yet another embodiment, at least one of the one or more permeation enhancers is an azone.

In an embodiment of the present invention, the pharmaceutical composition comprises one or more antioxidants in a total amount of about 0.01-3 wt % of the pharmaceutical composition. In another embodiment, the pharmaceutical composition comprises one or more antioxidants in a total amount of about 0.01-1.0 wt % of the pharmaceutical composition. the pharmaceutical composition comprises one or more antioxidants in a total amount of about 0.01-0.5 wt % of the pharmaceutical composition. In yet another embodiment, the one or more antioxidants is selected from the group consisting of tocopherol, butylated hyroxyanisole, and butylated hydroxytoluene.

In one embodiment of the present invention, the pharmaceutical composition comprises about 3-6 wt % of laquinimod, about 80-95 wt % pressure sensitive adhesive, and about 5-10 wt % permeation enhancers.

In one embodiment, the transdermal patch has a total area of about 5-50 cm². In another embodiment, the transdermal patch has a total area of about 5-30 cm². In another embodiment, the transdermal patch has a total area of about 5-20 cm². In another embodiment, the transdermal patch has a total area of about 5-10 cm². In another embodiment, the transdermal patch has a total area of 5 cm². In another embodiment, the transdermal patch has a total area of 10 cm². In another embodiment, the transdermal patch has a total area of 20 cm².

In one embodiment, the liner is a polyethylene terephthalate (PET) liner. In another embodiment, the PET liner is siliconized or has a fluoropolymeric coating. In yet another embodiment, the backing layer comprises a polymer selected from the group consisting of PET, polypropylene and polyurethane.

This invention also provides a method for delivering laquinimod across the skin of a subject comprising administering to the skin of the subject a transdermal patch as described herein.

This invention further provides a method for treating a human subject afflicted with a form of multiple sclerosis, comprising periodically administering to the human subject a transdermal patch as described herein.

This invention yet further provides a transdermal patch as described herein for use in treating a human subject afflicted with a form of multiple sclerosis.

For the foregoing embodiments, each embodiment disclosed herein is contemplated as being applicable to each of the other disclosed embodiments. In addition, the elements recited in the transdermal patch embodiments can be used in the method embodiments described herein and vice versa.

### Laquinimod

Laquinimod mixtures, compositions, and the process for the manufacture thereof are described in, e.g., U.S. Pat. No. 6,077,851, U.S. Pat. No. 7,884,208, U.S. Pat. No. 7,989,473, U.S. Pat. No. 8,178,127, U.S. Application Publication No. 2010-0055072, U.S. Application Publication No. 2012-0010238, and U.S. Application Publication No. 2012-0010239, each of which is hereby incorporated by reference in their entireties into this application.

Use of laquinimod for treating various conditions, and the corresponding dosages and regimens, are described in U.S. Pat. No. 6,077,851 (multiple sclerosis, insulin-dependent diabetes mellitus, systemic lupus erythematosus, rheumatoid arthritis, inflammatory bowel disease, psoriasis, inflammatory respiratory disorder, atherosclerosis, stroke, and Alzhemier's disease), U.S. Application Publication No. 2011-0027219 (Crohn's disease), U.S. Application Publication No. 2010-0322900 (Relapsing-remitting multiple sclerosis), U.S. Application Publication No. 2011-0034508 (Brain-derived neurotrophic factor (BDNF)-related diseases), U.S. Application Publication No. 2011-0218179 (Active lupus nephritis), U.S. Application Publication No. 2011-0218203 (Rheumatoid arthritis), U.S. Application Publication No. 2011-0217295 (Active lupus arthritis), and U.S. Application Publication No. 2012-0142730 (Reducing fatigue, improving quality of life, and providing neuroprotection in MS patients), each of which is hereby incorporated by reference in their entireties into this application.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

Laquinimod can be administered alone but is generally mixed with one or more pharmaceutically acceptable carriers. Laquinimod can be administered in admixture with suitable pharmaceutical diluents, extenders, excipients, or carriers (collectively referred to herein as a pharmaceutically acceptable carrier) suitably selected with respect to the intended form of administration (e.g., transdermal administration) and as consistent with conventional pharmaceutical practices.

The dosage unit can be in a form suitable for transdermal administration. Transdermal administration avoids hepatic metabolism and gastrointestinal degradation which can hinder effectiveness of orally administered drugs. However, the skin is not an absorptive organ and permeation of the drug to be administered is problematic. Other problems to be overcome include drug stability and formulation palatability.

General techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman at al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds).; Modern Pharmaceutics Drugs and the Pharmaceutical Sciences, Vol. 40 (Gilbert S. Banker, Christopher T. Rhodes, Eds). These references in their entireties are hereby incorporated by reference into this application.

### Terms

As used herein, and unless stated otherwise, each of the following terms shall have the definition set forth below.

As used herein, a “transdermal patch” can include, e.g., matrix patches and reservoir patches. Matrix patches contain the drug to be delivered in a semisolid matrix comprising drug and adhesive. Reservoir patches contain a layer, separate from the adhesive, which contains the drug to be delivered. In one embodiment, the transdermal patch as disclosed herein can have an area of between 5 to 20 cm². In another embodiment, the transdermal patch as disclosed herein can have an area of between 5 to 50 cm².

As used herein, a “needles patch” is a transdermal patch with small needles which micro-perforate the skin in order to increase permeation of the drug to be administered through the barrier. In an embodiment, the transdermal patch described herein is a needles patch.

As used herein, a “highly porous membrane” is a membrane having high gas, air and liquid permeability. Membrane parameters affecting permeability can be, e.g., total weight per surface area, thickness, porosity, mean flow pore size, and air permeability Gurley Number (a unit describing the number of seconds required for 100 cubic centimeters of air to pass through 1.0 square inch of a given material at a pressure differential of 4.88 inches of water (0.188 psi)(ISO 5636-5:2003)). A highly porous membrane can a SOLUPOR® membrane available from Lydall, Inc. (Manchester, Conn.).

Flux decreases with increasing Gurley number, and increasing membrane thickness. Other factors affecting flux include membrane pore size and weight per surface area. In addition, lower Gurley numbers are associated with higher risk of reservoir leakage. Parameters of suitable membranes should be selected such that 1) flux through the membrane is not affected, and 2) it provides an effective barrier to prevent reservoir liquid from leaking.

In one embodiment, the highly porous membrane has the following parameters: 1-20 g/m² total weight per surface area, 8-120 μm thickness, 40-99 vol. % porosity, optionally, 75-90 vol. % porosity, 1-200 s/50 ml Gurley Number, and up to 1.1 μm mean flow pore size. In another embodiment, the highly porous membrane has the following parameters: 3.0-16 g/m² total weight per surface area, 20-120 μm thickness, 80-90 vol. % porosity, 1-5 s/50 ml Gurley Number, and 0.3-1.1 μm mean flow pore size.

In another embodiment, the highly porous membrane has the following parameters: 40-50 vol. % porosity, 8-35 μm thickness, 4-20 g/m² basis weight, and 20-200 s/50 ml Gurley number, and <0.1 μm pore size. In yet another embodiment, the highly porous membrane has the following parameters: 75-90 vol. % porosity, 10-120 μm thickness, 3-20 g/m² basis weight, 1-100 s/50 ml Gurley number, and 0.05-1.0 μm pore size.

Parameters of some exemplary membranes are: 1) 3 g/m² total weight per surface area, 20 μm thickness, 83% porosity, 1.4 s/50 ml Gurley Number, and 0.7 μm mean flow pore size; 2) 5 g/m² total weight per surface area, 40 μm thickness, 86% porosity, 2 s/50 ml Gurley Number, and 1.1 μm mean flow pore size; 3) 7 g/m² total weight per surface area, 50 μm thickness, 85% porosity, 10 s/50 ml Gurley Number, and 0.3 μm mean flow pore size, 4) 7 g/m² total weight per surface area, 45 μm thickness, 84% porosity, 3 s/50 ml Gurley Number, and 0.7 μm mean flow pore size, 5) 10 g/m² total weight per surface area, 60 μm thickness, 83% porosity, 3 s/50 ml Gurley Number, and 0.5 μm mean flow pore size, 6) 16 g/m² total weight per surface area, 115 μm thickness, 85% porosity, 5 s/50 ml Gurley Number, and 0.5 μm mean flow pore size, and 7) 16 g/m² total weight per surface area, 120 μm thickness, 85% porosity, 4 s/50 ml Gurley Number, and 0.9 μm mean flow pore size.

As used herein, the term “composition”, as in a pharmaceutical composition, is intended to encompass a product comprising active ingredient(s) and inert ingredient(s) that make up the carrier, as well as any product which results, directly or indirectly from combination, complexation, or aggregation of two or more of the ingredients, or from dissociation of one or more of the ingredients, or from other types of reactions or interactions of one or more of the ingredients.

As used herein, a “pressure sensitive adhesive” or “PSA” is an adhesive which bonds when pressure is applied. Pressure sensitive adhesives include, but are not limited to acrylate copolymers such as Duro-Tak 87-4098, Duro-Tak 87-4098, Duro-Tak 87-2074, Duro-Tak 87-2510; Duro-Tak 87-2677; silicone adhesive, styrenic rubber adhesive and polyisobutylene adhesive. In an embodiment of the present invention, the PSA can be up to 90% by weight of the pharmaceutical composition or layer composition. In another embodiment of the present invention, the PSA can be up to 95% by weight of the pharmaceutical composition or layer composition.

As used herein, “permeation enhancers” are agents which increase bioavailability of the active ingredient. Permeation enhancers include, but are not limited to, fatty acids including oleic acid, propylene glycol, aloe vera oil, isopropyl myristate, n-Dodecyl nitrogen heterocyclic heptane-2-ketone, soya oil, diethylene glycol monoethyl ether (Transcutol®), alpha-tocopherol, alcohol (e.g., ethanol or isopropanol), sulfoxydes (e.g., dimethyl sulphoxyde), azones (e.g., lauryl caprolacton), pyrrolidone (and derivatives thereof), terpenes, terpenoides, ethyl acetate, methyl acetate, butyl acetate and cyclodextrines. In one embodiment, the permeation enhancer is oleic acid. In another embodiment, the permeation enhancer is isopropyl myristate. In yet another embodiment, the permeation enhancer is an azone. In one embodiment, the permeation enhancers can be up to 15% by weight of the pharmaceutical composition or layer composition.

As used herein, “antioxidant” refers to a compound that inhibits the oxidation of other molecules and includes, but is not limited to, tocopherol, BHA (butylated hydroxyanisole), butylated hydroxytoluene, a pharmaceutically acceptable salt or ester of the mentioned compounds, and mixtures thereof. In one embodiment, the antioxidant can be between 0.01 to 0.5% by weight of the pharmaceutical composition or layer composition. In another embodiment, the antioxidant can be up to about 3 wt % of the pharmaceutical composition or layer composition.

As used herein, a “backing layer” is an impervious flexible covering layer which protects the patch from the outside environment. A backing layer can be composed of a material such as a polymer including, but are not limited to, PET, polypropylene and polyurethane.

As used herein, a “perfusion enhancer” is an agent which increases blood flow to the capillary beds. Perfusion enhancers can include, but are not limited to, capsaicin and apitoxin.

As used herein and unless specified otherwise, “laquinimod” means laquinimod acid or a pharmaceutically acceptable salt thereof.

As used herein, an “amount” or “dose” of laquinimod as measured in milligrams refers to the milligrams of laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion.

As used herein, “saturation amount” of a substance in a composition means the amount above which the substance would no longer dissolve in the composition, and additional amounts of the substance will appear as a separate phase. Accordingly, where the composition as described herein contains a higher-than-saturation amount of laquinimod, the amount of laquinimod over the saturation amount will be present in the composition as non-dissolved laquinimod.

Administration of different amounts of laquinimod using transdermal patches of the present invention can be accomplished by applying one, two, three, four or more transdermal patches at the same time or consecutively or by applying a portion of a transdermal patch. For example ½ of a transdermal patch can be obtained by cutting a transdermal patch once and ¼ of a transdermal patch can be obtained by cutting a transdermal patch twice. Administration of an amount from about 0.1 to about 20 mg of laquinimod can be achieved using the transdermal patches of the present invention. For Example, administration of 2.5 mg laquinimod can be accomplished by applying ¼ of a transdermal patch containing 10 mg laquinimod and administration of 5 mg laquinimod can be accomplished by applying ½ of a transdermal patch containing 10 mg laquinimod.

As used herein, “about” in the context of a numerical value or range means±10% of the numerical value or range recited or claimed, and includes the numerical value or range recited or claimed.

As used herein, “effective” when referring to an amount of laquinimod refers to the quantity of laquinimod that is sufficient to yield a desired therapeutic response without undue adverse side effects (such as toxicity, irritation, or allergic response) commensurate with a reasonable benefit/risk ratio when used in the manner of this invention.

“Administering to the subject” or “administering to the (human) patient” means the giving of, dispensing of, or application of medicines, drugs, or remedies to a subject/patient to relieve, cure, or reduce the symptoms associated with a disease, disorder or condition, e.g., a pathological condition.

“Treating” as used herein encompasses, e.g., inducing inhibition, regression, or stasis of a disease or disorder, or lessening, suppressing, inhibiting, reducing the severity of, eliminating or substantially eliminating, or ameliorating a symptom of the disease or disorder.

“Inhibition” of disease progression or disease complication in a subject means preventing or reducing the disease progression and/or disease complication in the subject.

A “symptom” associated with a disease or disorder includes any clinical or laboratory manifestation associated with the disease or disorder and is not limited to what the subject can feel or observe.

As used herein, “a subject afflicted with” a disease, disorder or condition means a subject who has been clinically diagnosed to have the disease, disorder or condition.

As used herein, a subject at “baseline” is a subject prior to initiating laquinimod therapy.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-20 mg” includes 0.1 mg, 0.2 mg, 0.3 mg, etc. up to 20.0 mg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Experiment 1

### Laquinimod Free Acid and Laquinimod Sodium

### Manufacturing Laquinimod Free Acid

0.8 g Laquinimod-Na was dissolved in 75 ml water (HPLC grade). Under stirring, a solution of HCL (25%) was added drop-wise until no further precipitation occurred. The precipitated laquinimod free acid was filtered and then washed 3 times using water. The white residue was dried overnight under laminar flow at room temperature. The yield was 0.72 g corresponding to 96.0%.

Differential Scanning Calorimetry (DSC) Comparison: Laquinimod-Na Vs. Laquinimod

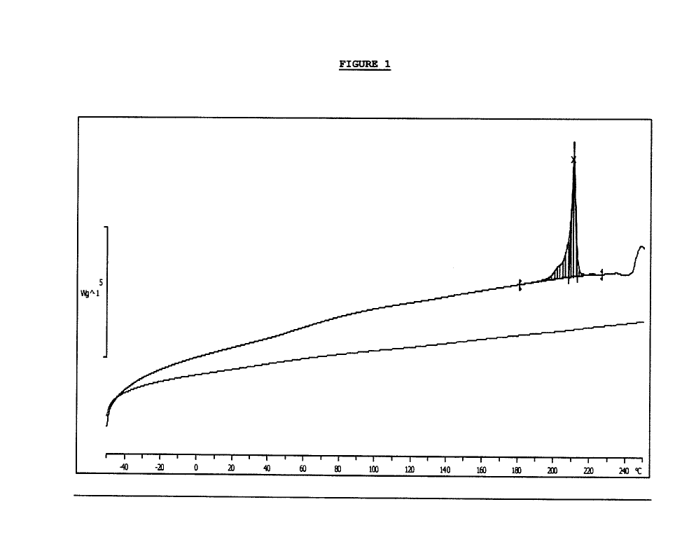

A DSC was measured in the range from −50° C. to 240° C. using a temperature rate of 10 K/min. The results are shown in FIG. 1. The melting point of laquinimod sodium (bottom thermogram) was found to be higher than 240° C. The melting point of laquinimod free acid (top thermogram) was determined at 206.43° C. (onset value).

### Laquinimod Free Acid Assay

The assay of laquinimod free acid was analyzed by HPLC, external standard calibration against laquinimod sodium standards. Assay Result: 100%.

### HPLC Method

Using the Synergy Polar RP-column, a polar embedded ether linked Phenylphase, a high retention for the aromatic structure was achieved. Laquinimod shows a good peak symmetry and is eluting at Rt 3.3 minutes at k′=3.5.

TABLE 1

| | |
|---|---|
| Pre column: | Phenomenex C8, PN: AJ0-4297 |
| Column: | Phenomenex Synergy Polar RP, 150 × 4 mm ID, |
| | 4 μm, |
| Flow: | 1.2 ml/Minute |
| Column temp. | 35° C. |
| Injection volume: | 5 μl for Assay, 5-25 μl skin for skin permeation |
| Mobile Phase | 10 mM KH₂PO₄ pH 6.0/Methanol, 50/50 |
| Detection: | DAD @ 230 nm, Ref. wavelength 450 nm |
| Pressure:: | approx. 170 bar |

### Test for Suitable Solvent-Solubilities

Saturation solubilities were measured in solvents typically used for incorporating of the drug into the pressure sensitive adhesives (PSA). Additional solubilities were measured in water, Phosphate buffered saline (PBS) Buffer (acceptor medium for skin permeation) and three different donor medium. All solubilities were measured at room temperature. Results are shown in Table 2 below:

TABLE 2

| Solvent/Solvent mixture | Solubility (μg/ml) |
|---|---|
| Ethanol/Dodecanol (95:5) | 4209 |
| Ethanol/Oleic acid (85:15) | 4785 |
| Propylenglycol/water (80:20) | 1281 |
| Water | 24 |
| PBS pH 7.4 | 1296 |
| Ethanol | 4098 |
| 2-Propanol | 1662 |
| Ethylacetate | 3909 |
| Acetone | 4884 |
| n-Heptane | 0.6 |

### Manufacturing Laquinimod Transdermal Therapeutic System (TTS)

Two batches were manufactured from two different PSA using Dodecanol as enhancer in each batch. The target dose in each formulation was 1.8 mg laquinimod free acid/10 cm².

### Batch 1

### Adhesive: DURO TAK 87-2677®

### Type: Acrylate-vinylacetate

Functional group: Carboxyl
Total Concentration of API and excipients: 55 mg/10 cm²

TABLE 3

| API and excipients | [%/DF] |
|---|---|
| Laquinimod free acid | 3.27 |
| DT-87-2677 (100% Polymer) | 90.91 |
| Dodecanol | 5.82 |
| Ethylacetate | N/A (substantially removed |
| | during drying process) |

### Batch 2

### Adhesive: DUROTAK 87-2074®

### Type: Acrylate

Functional group: Carboxyl/Hydroxyl
Total Concentration of API and excipients: 55 mg/10 cm²

TABLE 4

| API and excipients | [%/DF] |
|---|---|
| Laquinimod free acid | 3.27 |
| DT-87-2074 (100% Polymer) | 90.91 |
| Dodecanol | 5.82 |
| Ethylacetate | N/A (substantially removed |
| | during drying process) |

### Batch 3

A third batch was manufactured based on the Batch 2. The API concentration was doubled. Additionally Transcutol® HP was added to the formulation. Thus, the target dose was 3.6 mg laquinimod free acid/10 cm².

### Adhesive: DURO TAK 87-2074®

### Type: Acrylate

Functional groups: Carboxyl/Hydroxyl
Total Concentration of API and excipients: 60 mg/10 cm²

TABLE 5

| API and excipients | [%/DF] |
|---|---|
| Laquinimod free acid | 6.02 |
| DT-87-2074 | 83.61 |
| Dodecanol | 5.35 |
| Ethylacetate | N/A (substantially removed |
| | during drying process) |
| Transcutol HP | 5.02 |

### Batch 4

A reservoir TTS was manufactured as follows: The API was dissolved in the reservoir solution according to Table 6 at a concentration level of 10 mg/ml. The reservoir was made by sealing together a highly porous membrane (e.g., Solupor 10P05A, Lydall Inc.) and backing foil (e.g., Cotran 9733, 3M). The reservoir solution was filled into this reservoir using a syringe by inserting the injection needle into a small remaining, non-sealed area. After injecting 700 μl (corresponding to 7000 μg Laquinimod sodium) of the reservoir solution the needle was removed and the injection area was also sealed. The active area of 10 cm² was punched out at the outer side of the sealing using a punching tool.

TABLE 6

| Reservoir solution | % |
|---|---|
| Laquinimod sodium | 1% |
| Water | 31% |
| Ethanol | 38% |
| Laevulinic acid | 10% |
| Dimethylsulfoxide | 20% |

### Experiment 2

### Skin Permeation of Laquinimod Sodium Salt and Laquinimod Free Acid

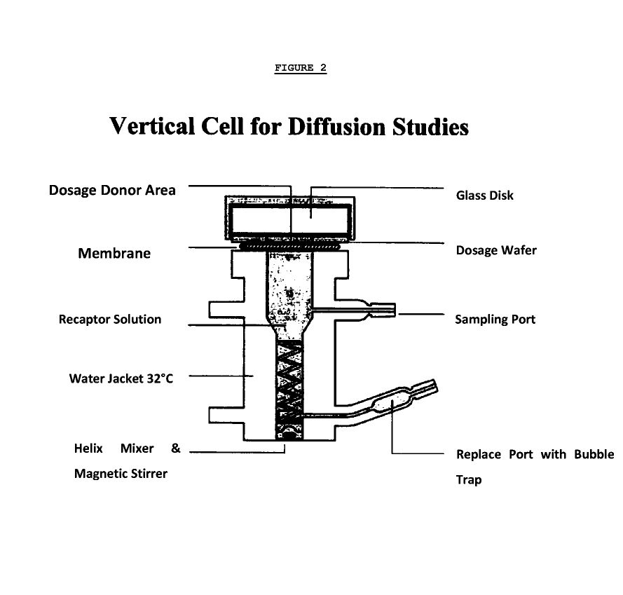

Transdermal permeation of laquinimod is tested by measuring permeation across human skin or hairless mouse skin by a diffusion cell system of Hanson Research consisting of acceptor cell and donor cell for laminate/TTS handling (Franz cell, FIG. 2) A description of the Franz cell system is provide by the product catalog published by Hanson Research (2001).

### Experiment 2.1

### Skin Permeation of Laquinimod Sodium Salt and Laquinimod Free Acid from Saturated Solutions—Mouse Skin (Screening I)

TABLE 7

| | |
|---|---|
| Cell: | Diffusion Cell System, Hanson Research, consisting |
| | of acceptor cell and original Hanson donor cell for |
| | liquid handling |
| Skin: | Hairless Mouse Skin 5-6 weeks old, female |
| Preparation: | 6 Placebo-rings (inner diameter 16 mm, outer diameter |
| | 25 mm) were punched out from a placebo laminate |
| | and after removing the release liner attached onto |
| | the skin. The mouse skin-placebo ring sandwiches |
| | were cut out at the outer diameter using a scalpel. |
| Donor | EtOH/Dodecanol 95/5 |
| Compositions: | EtOH/Oleic acid 85/15 |
| | PG/Water 8/2 |
| Donor Volume: | 2.0 ml, each containing undissolved API |
| Sampling Times: | 6, 9, 12, 18, 24 hours |
| Acceptor | PBS-Puffer pH 7.4 |
| Medium: | |
| Acceptor | 10 mL |
| Volume: | |
| Number of | n = 2 |
| cells per Donor: | |
| Permeation Area: | 1.77 cm² |
| Cell Temperature: | 32° C. ± 2.0° C. |
| Sampling: | Hanson AutoPlus/Microette Plus |

### Results for Laquinimod Sodium Salt

Applied Amounts of Laquinimod-Na onto the Skin

EtOH/Dodecanol 95/5, c=1.17 mg/ml, 2 ml saturated solution containing solid API.

EtOH/Oleic acid 85/15, c=4.30 mg/ml, 2 ml saturated solution containing solid API.

Propylene glycol (PG)/water 8/2, c=10.00 mg/ml, 2 ml saturated solution containing solid API.

TABLE 8
Permeated amounts in μg/cm²

| | EtOH/ | EtOH/oleic | EtOH/oleic | | |
|---|---|---|---|---|---|
| | Dodecanol | acid | acid | PG/water | PG/water |
| time (h) | 95/5 | 85/15 | 85/15 | 8/2 | 8/2 |
| 6 | 16.3 | 902.5 | 110.5 | 1.6 | 7.1 |
| 9 | 78.9 | 3287.1 | 589.2 | 4.7 | 3.3 |
| 12 | 262.3 | 4168.0 | 949.9 | 6.9 | 3.9 |
| 18 | 1386.6 | 4577.3 | 1445.8 | 10.5 | 4.7 |
| 24 | 2214.7 | 5015.1 | 2351.4 | 16.6 | 7.2 |

TABLE 9
Slopes in μg/(cm² × h)

| EtOH/ | | | | |
|---|---|---|---|---|
| Dodecanol | EtOH/oleic acid | EtOH/oleic acid | PG/water | PG/water |
| 95/5 | 85/15 | 85/15 | 8/2 | 8/2 |
| 20.9 | 794.9 | 159.6 | 1.0 | −1.3 |
| 61.1 | 293.6 | 120.2 | 0.7 | 0.2 |
| 187.4 | 68.2 | 82.6 | 0.6 | 0.1 |
| 138.0 | 73.0 | 150.9 | 1.0 | 0.4 |
| 20.9 | 794.9 | 159.6 | 1.0 | −1.3 |

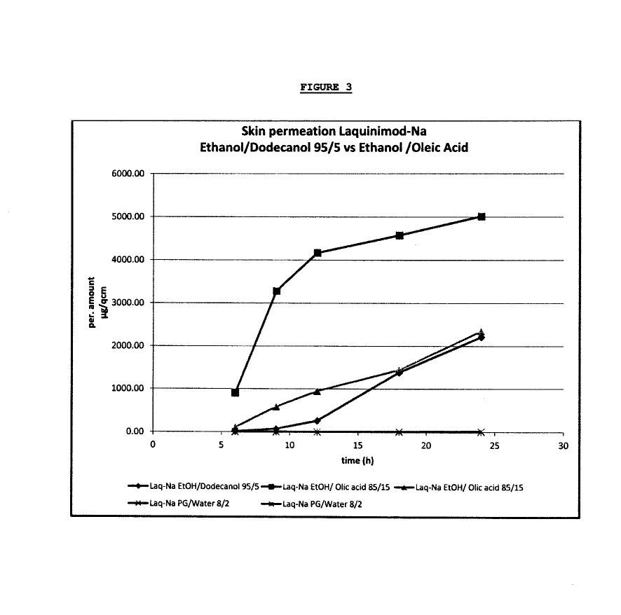

FIG. 3 shows the results of skin permeation of laquinimod sodium from saturated solutions (mouse skin). Skin of cell 1 (top curve) seems to have micro fissures.

### Results for Laquinimod Free Acid

Applied Amounts Laquinimod Free Acid onto the Skin

EtOH/Dodecanol 95/5, c=3.49 mg/ml, 2 ml saturated solution containing solid API.

EtOH/Oleic acid 85/15, c=3.93 mg/ml, 2 ml saturated solution containing solid API.

Propylene glycol (PG)/water 8/2, c=1.22 mg/ml, 2 ml saturated solution containing solid API.

TABLE 10
Permeated amounts in μg/cm²

| | EtOH/ | EtOH/ | EtOH/ | EtOH/ | PG/ | |
|---|---|---|---|---|---|---|
| time | Dodecanol | Dodecanol | oleic | oleic | water | PG/water |
| (h) | 95/5 | 95/5 | acid 85/15 | acid 85/15 | 8/2 | 8/2 |
| 6 | 7.1 | 17.9 | 236.0 | 48.5 | 0.6 | 1.1 |
| 9 | 206.2 | 81.8 | 880.6 | 119.4 | 2.3 | 1.9 |
| 12 | 688.0 | 171.3 | 1095.5 | 401.4 | 3.8 | 2.7 |
| 18 | 1505.3 | 405.3 | 1417.7 | 1033.5 | 6.4 | 4.1 |
| 24 | 2858.2 | 1165.6 | 2091.7 | 1715.6 | 11.2 | 7.5 |

TABLE 11
Slopes in μg/(cm² × h)

| EtOH/ | EtOH/ | EtOH/ | EtOH/ | | |
|---|---|---|---|---|---|
| Dodecanol | Dodecanol | oleic acid | oleic acid | PG/water | PG/water |
| 95/5 | 95/5 | 85/15 | 85/15 | 8/2 | 8/2 |
| 66.4 | 21.3 | 214.9 | 23.6 | 0.6 | 0.3 |
| 160.6 | 29.9 | 71.6 | 94.0 | 0.5 | 0.3 |
| 136.2 | 39.0 | 53.7 | 105.4 | 0.4 | 0.2 |
| 225.5 | 126.7 | 112.3 | 113.7 | 0.8 | 0.6 |

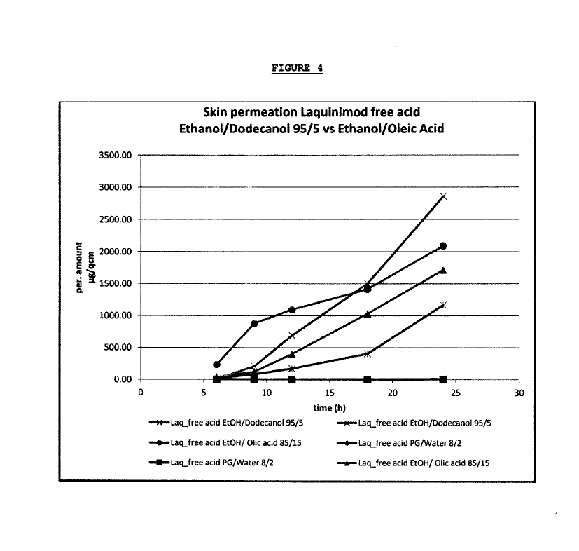

FIG. 4 shows the results of skin permeation of laquinimod free acid from saturated solutions (mouse skin).

### Experiment 2.2

### Skin Permeation of Laquinimod from TTS (Screening II)

Patch formulations were manufactured from two different PSA using Dodecanol as enhancer in each batch, the target dose in each formulation was 1.8 mg laquinimod free acid per 10 cm². The concentration of laquinimod is 3.3 wt %. If not otherwise described the permeation conditions comply with that described above for Screening Test I.

TABLE 12

| | |
|---|---|
| Cell: | Diffusion Cell System, Hanson Research, consisting of |
| | acceptor cell and original Hanson donor cell for |
| | laminate/TTS handling |
| Preparation: | 6 Placebo-rings (inner diameter 16 mm, outer diameter |
| | 25 mm) were punched out from a placebo laminate and |
| | after removing the release liner attached onto the skin. |
| | The mouse skin-placebo ring sandwiches were cut out |
| | at the outer diameter using a scalpel. |
| Donor: | Batch 1 (n = 2) and Batch 2 (n = 2) |
| Sampling | 1, 3, 6, 12, 18, 24 hours |
| Times: | |

### Results for Skin Permeation of Laquinimod Transdermal Therapeutic System (TTS)

TABLE 13
Permeated amounts in μg/cm²

| time (h) | Batch 1 | Batch 1 | Batch 2 | Batch 2 |
|---|---|---|---|---|
| 1 | 0.1 | 0.1 | 0.1 | 0.1 |
| 3 | 0.6 | 0.2 | 0.5 | 0.4 |
| 6 | 1.5 | 0.5 | 1.6 | 1.4 |
| 12 | 3.5 | 1.2 | 4.2 | 4.1 |
| 18 | 6.2 | 2.8 | 7.6 | 8.1 |
| 24 | 9.5 | 4.4 | 11.4 | 12.2 |

TABLE 14
Slopes in μg/(cm² × h)

| Batch 1 | Batch 1 | Batch 2 | Batch 2 |
|---|---|---|---|
| 0.2 | 0.0 | 0.2 | 0.2 |
| 0.3 | 0.1 | 0.4 | 0.3 |
| 0.3 | 0.1 | 0.4 | 0.4 |
| 0.5 | 0.3 | 0.6 | 0.7 |
| 0.5 | 0.3 | 0.6 | 0.7 |

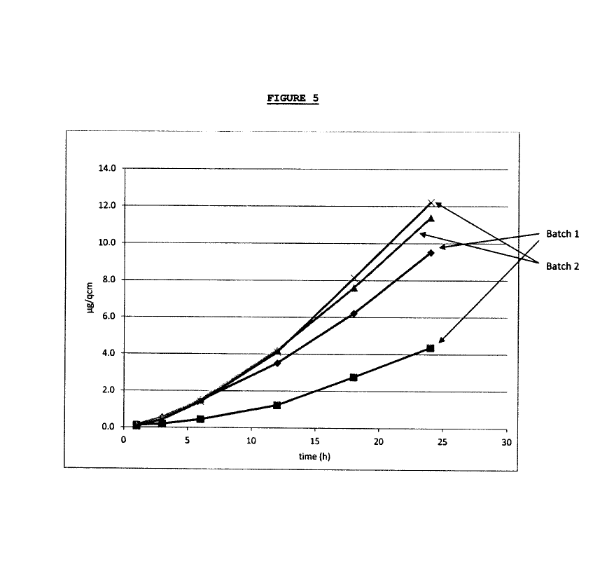

FIG. 5 shows the results of skin permeation of laquinimod free acid from TTS in hairless mouse skin (Batches 1 and 2).

### Experiment 2.3

### Correlation Skin Permeation Results—Hairless Mouse Skin/Human Skin (Screening III)

TABLE 15

| | |
|---|---|
| Cell: | Diffusion Cell System, Hanson Research, consisting of |
| | acceptor cell and original donor cell for laminate/TTS |
| | handling |
| Skin: | Cell 1/2: Batch 3 (Human Skin) |
| | Cell 3/4: Batch 3 spiked with oleic acid** (Human Skin) |
| | Cell 5/6: Batch 3 (Mouse Skin) |
| Donor Volume: | 0.9 ml |
| Sampling | 1, 3, 6, 72 hours (between 6 and 72 hours no samples |
| Times: | were taken due to a failure of the automatic sampler) |

**Spiking Procedure: both TTS were spiked with 100 μL of an ethanolic solution of oleic acid (containing 15% oleic acid). After removing the ethanol by drying for 30 minutes at 65° C. a thin film of oleic acid remained on the adhesive surface. The amount of oleic acid applied onto the patch corresponds to 12.9 mg oleic acid/1.77 cm².

### Results

TABLE 16
permeated amounts in μg/cm²

| | | | Human | Human | | |
|---|---|---|---|---|---|---|
| | | | skin + | skin + | | |
| time | Human | Human | 100 μl oleic | 100 μl oleic | Mouse | Mouse |
| (h) | skin | skin | acid 15%) | acid 15%) | skin | skin |
| 1 | 0.4 | 0.5 | 0.4 | 0.2 | 1.3 | 2.3 |
| 3 | 0.5 | 0.6 | 0.7 | 1.2 | 5.0 | 7.4 |
| 6 | 2.4 | 2.4 | 5.3 | 5.5 | 15.8 | 18.3 |
| 72 | 46.4 | 39.6 | 99.1 | 61.8 | 141.5 | 134.5 |

TABLE 17
Slopes in μg/(cm² × h)

| | | Human | Human | | |
|---|---|---|---|---|---|
| Human | Human | skin + 100 μl | skin + 100 μl | Mouse | Mouse |
| skin | skin | oleic acid 15%) | oleic acid 15%) | skin | skin |
| 0.1 | 0.0 | 0.1 | 0.5 | 1.9 | 2.6 |
| 0.6 | 0.6 | 1.5 | 1.4 | 3.6 | 3.6 |
| 0.7 | 0.6 | 1.4 | 0.9 | 1.9 | 1.8 |

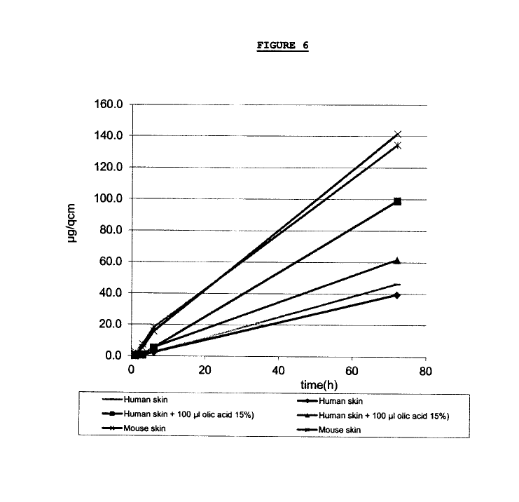

FIG. 6 shows skin permeation (human skin vs. mouse skin) for laquinimod free acid from TTS Batch 3.

### Experiment 2.4

### Skin Permeation of a Reservoir Patch (Batch 4) Through Human Abdominal Skin

A permeation study with Batch 4 (Table 6) was performed using abdominal human skin, dermatomised to a thickness of approximately 500 μm. For this purpose 2 cells were prepared according to Table 7 applying 700 μl reservoir solution directly onto the skin (Sample 1 & 2).

2 more cells were prepared by attaching each one reservoir patch onto one skin (Sample 3 & 4). The results are shown in Table 18.

TABLE 18
permeated amount (μg/cm2)

| | | | Sample 3 + | Sample 4 + |
|---|---|---|---|---|
| time | Sample | Sample | Solupor | Solupor |
| (h) | 1 | 2 | Membrane | Membrane |
| 1.00 | 2.24 | 17.86 | 11.24 | 7.77 |
| 3.00 | 26.59 | 49.10 | 39.45 | 23.23 |
| 6.00 | 77.39 | 119.81 | 99.74 | 56.42 |
| 12.00 | 175.26 | 246.96 | 209.74 | 131.44 |
| 18.00 | 265.54 | 345.41 | 270.06 | 190.86 |
| 24.00 | 308.98 | 389.16 | 292.01 | 218.81 |

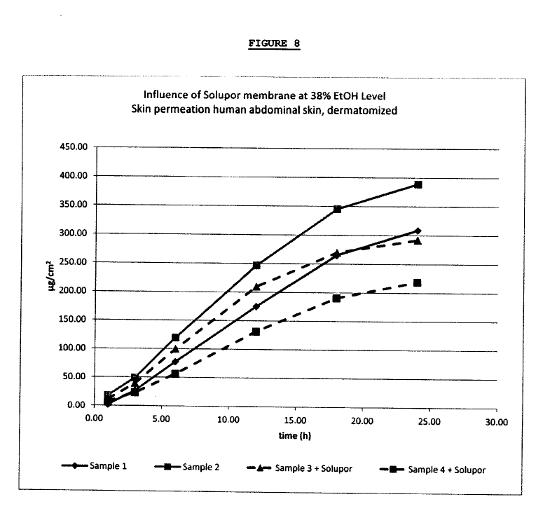

FIG. 8 shows the skin permeation (human abdominal skin, dermatomized) for Laquinimod sodium salt from reservoir patch Batch 4.

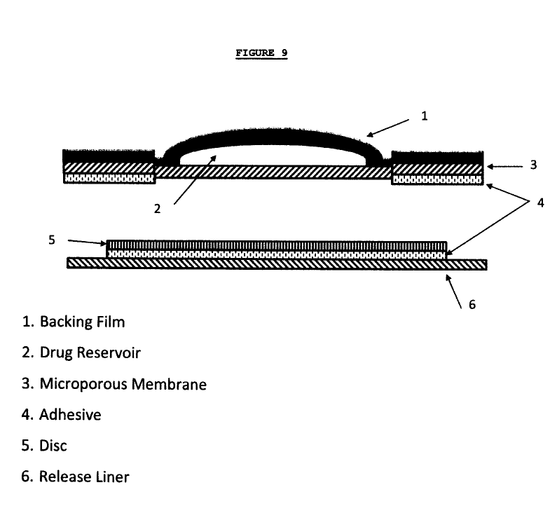

FIG. 9 schematically shows the structure of the reservoir patch. Release line (6) as well as adhered disc (5) is removed before application.

### DISCUSSION

The correlation of the permeated amount (μg/cm²) mouse skin/human skin for laquinimod was found to be a factor 11.3 after 3 hours, 7.1 after 6 hours and 3.2 after 72 h hours.

The correlation of the flux (μg/cm²×h) mouse skin/human skin for laquinimod was found to be a factor 30 after 3 hours, and 6 after 6 hours.

The addition of oleic acid showed a significant increase of the permeated amount (μg/cm²). The increase was a factor 2.2 after 6 hours and 1.9 after 72 hours.

The screening experiments I, II and III indicate that the use of oleic acid leads to an increase of laquinimod concentration, especially within the first 6 hours of application.

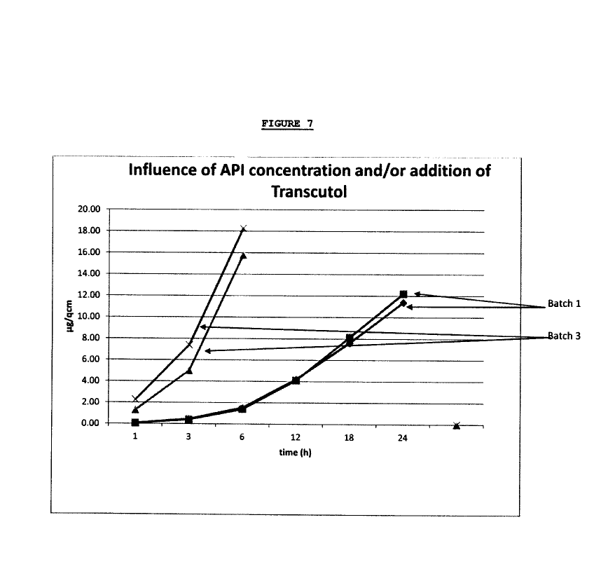

FIG. 7 (skin permeation of hairless mouse skin—laquinimod free acid from TTS, Batch 1 (1.6 mg/10 cm²) and Batch 3 (3.2 mg/10 cm²) shows the comparison of the in vitro skin permeation test results through hairless mouse skin from two batches containing different drug concentrations and different enhancers. It is likely that the higher flux of Batch 3 is achieved by the doubled API concentration.

However, contrary to the prediction provided by Fick's law, the increase in flux is not proportional to the increase in API concentration. Rather, by doubling the concentration of laquinimod, the flux more than doubled. This result is unexpected.

Without being bound by theory, the inventors believe that the increase in permeation is a result of the composition being supersaturated by laquinimod. When supersaturated, a portion of laquinimod which is not dissolved in the composition is present as laquinimod crystals. Upon contact with the skin, the laquinimod crystals dissolves into the skin rather than stay in the already-saturated pharmaceutical composition. Accordingly, in one embodiment of the present invention, the amount of laquinimod present in the pharmaceutical composition is a least laquinimod's saturation amount. In another embodiment, the amount of laquinimod present in the pharmaceutical composition is higher than laquinimod's saturation amount (the pharmaceutical composition is supersaturated with laquinimod).

FIG. 8 (skin permeation of a reservoir patch—laquinimod sodium, Batch 4 (7 mg/10 cm²)) shows the in vitro skin permeation test results of a reservoir patch (TRS) through human skin in comparison to the plain reservoir solution. This results show that the membrane of the TRS has no significant influence on the flux. The reservoir solution contains among other ingredients 38% ethanol. The placebo formulation showed no skin irritation within wearer trials. The TRS could be fixed onto the skin by using an adhesive ring from an ethanol resistant pressure sensitive adhesive, e.g. a silicone adhesive. The flux of the active ingredient increases with increasing laquinimod concentration in the reservoir solution up to its saturation solubility. Simultaneously the volume of the reservoir solution should be decreased for limitation of the absolute active ingredient content in the TRS.

### REFERENCES

1. Bjartmar and Fox (2002) “Pathological mechanisms and disease progression of multiple sclerosis: therapeutic implication”, Drugs of Today. 38:7-29.
2. Brex et al. (2002) “A longitudinal study of abnormalities on MRI and disability from multiple sclerosis”, N Engl J Med. Jan. 17, 2002 346(3):158-64.
3. Brück (2011) “Insight into the mechanism of laquinimod action.” J Neurol Sci. 2011 Jul. 15; 306(1-2):173-9.
4. Brück et al. (2012) “Reduced astrocytic NF-kappaB activation by laquinimod protects from cuprizone-induced demyelination.” Acta Neuropathol. 124:411-424.
5. Brunmark et al. (2002) “The new orally active immunoregulator laquinimod (ABR-215062) effectively inhibits development and relapses of experimental autoimmune encephalomyelitis.” J Neuroimmunol. 130:163-172.
6. Comi et al. (2008) “Effect of laquinimod on MRI-monitored disease activity in patients with relapsing-remitting multiple sclerosis: a multicentre, randomised, double-blind, placebo-controlled phase IIb study”. Lancet. 371:2085-2092.
7. Comi et al. (2012) “Placebo-controlled trial of oral laquinimod for multiple sclerosis.” N Engl J Med. 366:1000-1009.
8. De Stefano et al. (1999) “Evidence of early axonal damage in patients with multiple sclerosis”, Neurology. 52(Suppl 2):A378.
9. De Stefano et al. (2003) “Evidence of early cortical atrophy in MS: relevance to white matter changes and disability.” Neurology. 60:1157-1162.
10. Dunitz. M. (1999) Multiple sclerosis therapeutics, Ed. Rudick and Goodkin. London: Taylor & Francis, 1999.
11. Frohman et al. (2003) “The utility of MRI in suspected MS: report of the Therapeutics and Technology Assessment Subcommittee of the American Academy of Neurology”, Neurology. Sep. 9, 2003, 61(5):602-11.
12. Gasperini and Ruggieri (2009) “New oral drugs for multiple sclerosis.” Neurol Sci. 30(Suppl 2):S179-183.
13. Gurevich et al. (2010) “Laquinimod suppress antigen presentation in relapsing-remitting multiple sclerosis: in-vitro high-throughput gene expression study.” J Neuroimmunol. 221:87-94.
14. Hohlfeld et al. (2000) “The neuroprotective effect of inflammation: implications for the therapy of multiple sclerosis”, J Neuroimmunol. 107:161-166.
15. Kurtzke J F. (1983) “Rating neurologic impairment in multiple sclerosis: an expanded disability status scale (EDSS)”, Neurology 33(11):1444-1452.
16. McDonald, (2001) “Guidelines from the International Panel on the Diagnosis of Multiple Sclerosis” Ann. Neurol. 50:121-127.
17. Multiple sclerosis: its diagnosis, symptoms, types and stages, 2003, albany.net/.about.tjc/multiple-sclerosis.html; What are the Types of Multiple Sclerosis?, 2005, <imaginis.com/multiple-sclerosis/types-of-ms.asp? mode=1>.
18. National Multiple Sclerosis Society Website “What We Know About Progressive-Relapsing MS (PRMS).” 28 Jan. 2013 Web. <http://www.nationalmssociety.org/about-multiple-sclerosis/progressive-ms/progressive-relapsing-ms/index.aspx>.
19. Neuhaus et al. (2003) “Immunomodulation in multiple sclerosis: from immunosuppression to neuroprotection”, Trends Pharmacol Sci. 24:131-138.
20. Noseworthy et al. (2000) “Multiple sclerosis”, N Engl J Med. 343:938-952.
21. PCT International Application Publication No. WO 2005/074899, published Aug. 18, 2005 (Jansson et al.).
22. Polman et al. (2005) “Diagnostic criteria for multiple sclerosis: 2005 revisions to the McDonald Criteria”, Annals of Neurology, 58(6):840-846.
23. Polman et al. (2005) “Treatment with laquinimod reduces development of active MRI lesions in relapsing MS.” Neurology. 64:987-991.
24. Polman et al. (2006) “A randomized, placebo-controlled trial of natalizumab for relapsing multiple sclerosis”, N Eng J Med. 354:899-910.
25. Poser et al. (1983) “New Diagnostic Criteria for Multiple Sclerosis: Guidelines for Research Protocols”, Annals of Neurology, March 1983, 13(3):227-230.
26. Product catalog published by Hanson Research (2001) (<http://www.prosense.net/files/MicroettePlus.pdf>, retrieved on Feb. 27, 2013).
27. Results of Phase III BRAVO Trial Reinforce Unique Profile of Laquinimod for Multiple Sclerosis Treatment; Teva Pharma, Active Biotech Post Positive Laquinimod Phase 3 ALLEGRO Results.
28. RTT News Article dated Apr. 12, 2011, entitled “Teva Pharma, Active Biotech Post Positive Laquinimod Phase 3 ALLEGRO Results”.
29. Runström et al. (2006) “Inhibition of the development of chronic experimental autoimmune encephalomyelitis by laquinimod (ABR-215062) in IFN-β k.o. and wild type mice” Journal of Neuroimmunology, 173(2006):69-78.
30. Sandberg-Wollheim et al. (2005) “48-week open safety study with high-dose oral laquinimod in patients”, Mult Scler. 11:S154 (Abstract).
31. Spain et al. (2009) “Recent developments in multiple sclerosis therapeutics.” BMC Medicine. 7:74.
32. The National MS Society (USA), The Disease Modifying Drug Brochure, Oct. 19, 2006.
33. Types of Multiple Sclerosis (MS), 2005, <themcfox.com/multiple-sclerosis/types-of-ms/types-of-multiple-sclerosis.htm>.
34. U.S. Patent Application Publication No. 2010-0322900, published Dec. 23, 2010 (Tarcic et al.).
35. U.S. Patent Application Publication No. 2011-0027219, published Feb. 3, 2011 (Tarcic et al.).
36. U.S. Patent Application Publication No. 2011-0034508, published Feb. 10, 2011 (Liat Hayardeny).
37. U.S. Patent Application Publication No. 2011-0217295, published Sep. 8, 2011 (Haviv and Tarcic).
38. U.S. Patent Application Publication No. 2011-0218179, published Sep. 8, 2011 (Haviv and Tarcic).
39. U.S. Patent Application Publication No. 2011-0218203, published Sep. 8, 2011 (Joel Kaye et al.).
40. U.S. Patent Application Publication No. 2012-0010238, published Jan. 12, 2012 (Fristedt).
41. U.S. Patent Application Publication No. 2012-0010239, published Jan. 12, 2012 (Piryatinsky et al.).
42. U.S. Patent Application Publication No. 2012-0142730, published Jun. 7, 2012 (Tarcic et al.).
43. U.S. Pat. No. 6,077,851, issued Jun. 20, 2000 (Bjork et al).
44. U.S. Pat. No. 7,589,208, issued Sep. 15, 2009 (Jansson et al).
45. U.S. Pat. No. 7,884,208, issued Feb. 8, 2011 (Frenkel et al.).
46. U.S. Pat. No. 7,989,473, issued Aug. 2, 2011 (Patashnik et al.).
47. U.S. Pat. No. 8,178,127, issued May 15, 2012 (Safadi et al.).
48. U.S. Pat. No. 8,252,993, issued Aug. 28, 2012 (Gant and Shahbaz).
49. Vollmer at al. (2011) “A placebo-controlled and active comparator phase III trial (BRAVO) for relapsing-remitting multiple sclerosis. 5th Joint Triennial Congress of the European and Americas Committees for Treatment and Research in Multiple Sclerosis.” Oct. 19-22, 2011; Amsterdam, The Netherlands: 148.
50. Wegner et al. (2010) “Laquinimod interferes with migratory capacity of T cells and reduces IL-17 levels, inflammatory demyelination and acute axonal damage in mice with experimental autoimmune encephalomyelitis.” J Neuroimmunol 227:133-43.
51. Yang et al. (2004) “Laquinimod (ABR-215062) suppresses the development of experimental autoimmune encephalomyelitis, modulates the Th1/Th2 balance and induces the Th3 cytokine TGF-beta in Lewis rats.” J Neuroimmunol. 156:3-9.

What is claimed is:

1. A transdermal patch comprising:
a) a backing layer;
b) a liner;
c) optionally, a highly porous membrane; and
d) a pharmaceutical composition comprising:
(i) optionally, a pressure sensitive adhesive in an amount of up to about 95 wt % of the pharmaceutical composition,
(ii) laquinimod in an amount of about 0.1-20 wt % of the pharmaceutical composition, and
(iii) optionally, one or more permeation enhancers in a total amount of up to about 70 wt % of the pharmaceutical composition.

2. The transdermal patch of claim 1, wherein the pharmaceutical composition is in the form of a layer, film, or liquid.

3. The transdermal patch of claim 1 or 2, in the form of a matrix patch, wherein the pharmaceutical composition further comprises a pressure sensitive adhesive in an amount of up to about 95 wt % of the pharmaceutical composition.

4. The transdermal patch of claim 1 or 2, in the form of a reservoir patch and further comprising a highly porous membrane.

5. The transdermal patch of any one of claims 1-4, wherein laquinimod is laquinimod free acid.

6. The transdermal patch of any one of claims 1-4, wherein laquinimod is laquinimod sodium.

7. The transdermal patch of any one of claims 1-6, wherein the amount of laquinimod present in the pharmaceutical composition is a least laquinimod's saturation amount.

8. The transdermal patch of claim 7, wherein the amount of laquinimod present in the pharmaceutical composition is higher than laquinimod's saturation amount.

9. The transdermal patch of any one of claims 1-8, wherein laquinimod is present in an amount of about 1-15 wt % of the pharmaceutical composition.

10. The transdermal patch of claim 9, wherein laquinimod is present in an amount of about 2-10 wt % of the pharmaceutical composition.

11. The transdermal patch of claim 10, wherein laquinimod is present in an amount of about 1 wt % of the pharmaceutical composition.

12. The transdermal patch of claim 10, wherein laquinimod is present in an amount of about 3.3 wt % of the pharmaceutical composition.

13. The transdermal patch of claim 10, wherein laquinimod is present in an amount of about 6.0 wt % of the pharmaceutical composition.

14. The transdermal patch of any one of claims 1-13, containing about 0.1-20 mg laquinimod.

15. The transdermal patch of claim 14, containing about 6-8 mg laquinimod.

16. The transdermal patch of any one of claims 1-15, wherein the pressure sensitive adhesive is present in an amount of about 80-95 wt % of the pharmaceutical composition.

17. The transdermal patch of any one of claims 1-16, wherein the pressure sensitive adhesive comprises an acrylate copolymer.

18. The transdermal patch of any one of claims 1-17, wherein the one or more permeation enhancers is present in a total amount of up to about 70 wt % of the pharmaceutical composition.

19. The transdermal patch of claim 18, wherein the one or more permeation enhancers is present in a total amount of up to about 20 wt % of the pharmaceutical composition.

20. The transdermal patch of claim 19, wherein the one or more permeation enhancers is present in a total amount of up to about 15 wt % of the pharmaceutical composition.

21. The transdermal patch of any one of claims 1-20, wherein the one or more permeation enhancers is selected from the group consisting of a fatty acid, an alcohol, diethylene glycol monoethyl ether, alpha-tocopherol, a sulfoxyde, an azone, a pyrrolidone or a derivative thereof, a terpene, a terpenoide, methyl acetate, butyl acetate and a cyclodextrine.

22. The transdermal patch of claim 21, wherein at least one of the one or more permeation enhancers is oleic acid.

23. The transdermal patch of claim 21, wherein at least one of the one or more permeation enhancers is isopropyl myristate.

24. The transdermal patch of claim 21, wherein at least one of the one or more permeation enhancers is an azone.

25. The transdermal patch of claim 21, wherein at least one of the one or more permeation enhancers is ethanol.

26. The transdermal patch of any one of claims 1-25, wherein the pharmaceutical composition comprises one or more antioxidants in a total amount of about 0.01-3 wt % of the pharmaceutical composition.

27. The transdermal patch of claim 26, wherein the one or more antioxidants is selected from the group consisting of tocopherol, butylated hyroxyanisole, and butylated hydroxytoluene.

28. The transdermal patch of any one of claims 1-3, wherein the pharmaceutical composition comprises about 3-6 wt % of laquinimod, about 80-95 wt % pressure sensitive adhesive, and about 5-10 wt % permeation enhancers.

29. The transdermal patch of claim 4, wherein the pharmaceutical composition comprises about 1 wt % of laquinimod, about 30-35 wt % water and about 65-70 wt % permeation enhancers.

30. The transdermal patch of any one of claims 1-29, having a total area of about 5-50 cm².

31. The transdermal patch of any one of claims 1-30, wherein the liner is a polyethylene terephthalate (PET) liner.

32. The transdermal patch of claim 30 wherein the PET liner is siliconized or has a fluoropolymeric coating.

33. The transdermal patch of any one of claims 1-30, wherein the backing layer comprises a polymer selected from the group consisting of PET, polypropylene and polyurethane.

34. A method for delivering laquinimod across the skin of a subject comprising administering to the skin of the subject a transdermal patch of any one of claims 1-30.

35. A method for treating a human subject afflicted with a form of multiple sclerosis, comprising periodically administering to the human subject a transdermal patch of any one of claims 1-30.

36. A transdermal patch of any one of claims 1-30 for use in treating a human subject afflicted with a form of multiple sclerosis.
